(19) United States
(12) Patent Application Publication
(10) Pub. No.: US 2013/0218274 A1
(43) Pub. Date: Aug. 22, 2013

(54) SHREDDED TISSUE GRAFTS AND METHODS FOR MAKING AND USING THE SAME

(71) Applicant: MIMEDX GROUP, INC., (US)

(72) Inventors: Randall Spencer, Kennesaw, GA (US); Steven Ray, Kennesaw, GA (US)

(73) Assignee: MIMEDX GROUP, INC., Kennesaw, GA (US)

(21) Appl. No.: 13/688,091

(22) Filed: Nov. 28, 2012

Related U.S. Application Data
(60) Provisional application No. 61/566,053, filed on Dec. 2, 2011.

(51) Int. Cl. A61F 2/10; A61F 2/02

(52) U.S. Cl. CPC A61F 2/105; A61F 2/02
USPC 623/15.12; 623/23.72

(57) ABSTRACT

Described herein are shredded tissue grafts composed of one or more placental components. The compositions have numerous medical applications with respect to wound healing and complications associated with wound healing. Methods for making the shredded tissue graft compositions are also described herein.

### CROSS-REFERENCE TO RELATED APPLICATION

This application claims the benefit under 35 U.S.C. §119(e) of U.S. Provisional Application No. 61/566,053, filed Dec. 2, 2011, which is incorporated herein by reference in its entirety.

### BACKGROUND

Most surgical wounds are closed by primary intention. However, in certain circumstances, it is desirable for the wound to heal by secondary intention as well. The procedure for facilitating secondary intention generally involves repeated packing and dressing of the wound. Many surgeons still use traditional soaked gauze for dressing and packing open surgical wounds and cavities. The use of gauze for packing wounds provides several complications including pain associated with the removal of the gauze from the wound and the threat of possible infection. Moreover, gauze does not promote healing of the wound. Although alternatives to gauze are currently available for use in wound packing applications, they are not ideal with respect to wound healing and the complications associated with wound healing.

### SUMMARY

Described herein are shredded tissue grafts composed of one or more placental components. The compositions have numerous medical applications with respect to wound healing and complications associated with wound healing. Methods for making the shredded tissue graft compositions are also described herein.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 is an overview flow chart of the process for making the shredded tissue graft compositions described herein.

FIG. 2 shows an exemplary tissue graft composed of amnion membrane and chorion useful in making the shredded tissue grafts.

### DETAILED DESCRIPTION

Before the present articles and methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings.

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not. For example, the phrase “optionally cleaning step” means that the cleaning step may or may not be performed.

The term “subject” as used herein is any vertebrate organism.

The term “amnion membrane” as used herein includes amniotic tissue where the intermediate tissue layer has not been removed.

The term “amnion” as used herein includes amnion tissue where the intermediate tissue layer has been removed.

Titles or subtitles may be used in the specification for the convenience of a reader, which are not intended to influence the scope of the present invention. Additionally, some terms used in this specification are more specifically defined below.

### I. Shredded Tissue Grafts and Methods for Making Thereof

Described herein are shredded tissue grafts and methods of making and using thereof. The tissue grafts described herein are composed of one or more placental components. The term “placental components” as used herein include any materials or tissues present in the placenta and the umbilical cord. In one aspect, placental components include, but are not limited to, amnion membrane, amnion, chorion, Wharton's jelly, placental disk, or any combination thereof.

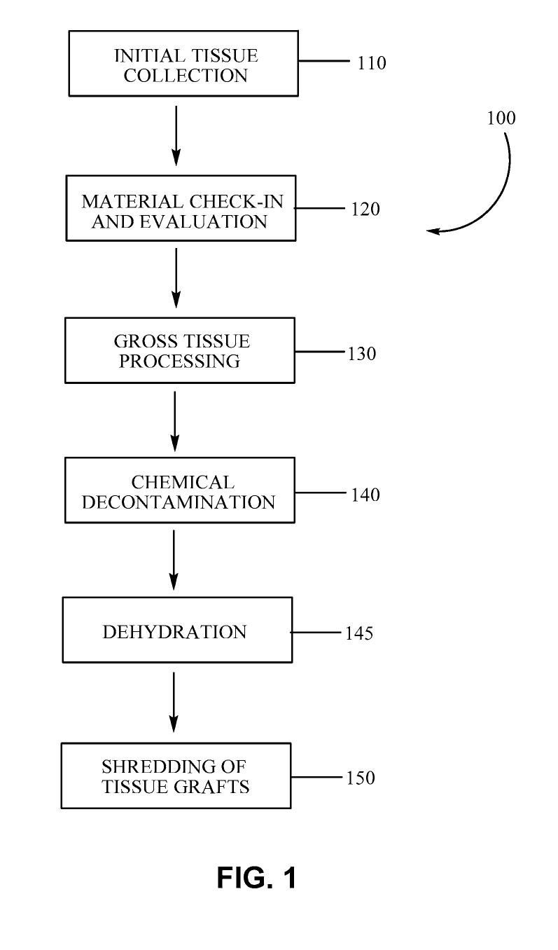

In some aspects, the shredded tissue grafts are multilayered systems composed of one or more placental components laminated to one another. FIG. 1 depicts an exemplary overview (100) and certain aspects of the steps to harvest, process, and prepare placental components for use in the preparation of the shredded tissue grafts described herein. More detailed descriptions and discussion regarding each individual step will follow. Initially, the placental tissue is collected (step 110). The material is preserved and transported in conventional tissue preservation manner to a suitable processing location or facility for check-in and evaluation (step 120). Gross processing, handling, and separation of the tissue layers then takes place (step 130). Acceptable tissue is then decontaminated (step 140). After decontamination, the placental components (e.g., amnion membrane, amnion, chorion, Wharton's jelly, placental disk) are dehydrated (step 145) and subsequently shredded to produce the shredded tissue grafts (step 150). Each step is described in detail below.

### Initial Tissue Collection (Step 110)

The components used to produce the tissue grafts are derived from the placenta. The source of the placenta can vary. In one aspect, the placenta is derived from a mammal such as human and other animals including, but not limited to, cows, pigs, and the like can be used herein. In the case of humans, the recovery of the placenta originates in a hospital, where it is collected during a Cesarean section birth. The donor, referring to the mother who is about to give birth, voluntarily submits to a comprehensive screening process designed to provide the safest tissue possible for transplantation. The screening process preferably tests for antibodies to the human immunodeficiency virus type 1 and type 2 (anti-HIV-1 and anti-HIV-2), antibodies to the hepatitis B virus (anti-HBV) hepatitis B surface antigens (HBsAg), antibodies to the hepatitis C virus (anti-HCV), antibodies to the human T-lymphotropic virus type I and type II (anti-HTLV-I, anti-HTLV-II), CMV, and syphilis, and nucleic acid testing for human immune-deficiency virus type 1 (HIV-1) and for the hepatitis C virus (HCV), using conventional serological tests. The above list of tests is exemplary only, as more, fewer, or different tests may be desired or necessary over time or based upon the intended use of the grafts, as will be appreciated by those skilled in the art.

Based upon a review of the donor's information and screening test results, the donor will either be deemed acceptable or not. In addition, at the time of delivery, cultures are taken to determine the presence of bacteria, for example, Clostridium or Streptococcus. If the donor's information, screening tests, and the delivery cultures are all satisfactory (i.e., do not indicate any risks or indicate acceptable level of risk), the donor is approved by a medical director and the tissue specimen is designated as initially eligible for further processing and evaluation.

Human placentas that meet the above selection criteria are preferably bagged in a saline solution in a sterile shipment bag and stored in a container of wet ice for shipment to a processing location or laboratory for further processing.

If the placenta is collected prior to the completion or obtaining of results from the screening tests and delivery cultures, such tissue is labeled and kept in quarantine. The placenta is approved for further processing only after the required screening assessments and delivery cultures, which declare the tissue safe for handling and use, are satisfied and obtains final approval from a medical director.

### Material Check-in and Evaluation (Step 120)

Upon arrival at the processing center or laboratory, the shipment is opened and verified that the sterile shipment bag/container is still sealed and in the coolant, that the appropriate donor paperwork is present, and that the donor number on the paperwork matches the number on the sterile shipment bag containing the tissue. The sterile shipment bag containing the tissue is then stored in a refrigerator until ready for further processing.

### Gross Tissue Processing (Step 130)

When the tissue is ready to be processed further, the sterile supplies necessary for processing the placental tissue further are assembled in a staging area in a controlled environment and are prepared for introduction into a controlled environment. If the controlled environment is a manufacturing hood, the sterile supplies are opened and placed into the hood using conventional sterile technique. If the controlled environment is a clean room, the sterile supplies are opened and placed on a cart covered by a sterile drape. All the work surfaces are covered by a piece of sterile drape using conventional sterile techniques, and the sterile supplies and the processing equipment are placed onto the sterile drape, again using conventional sterile techniques.

Processing equipment is decontaminated according to conventional and industry-approved decontamination procedures and then introduced into the controlled environment. The equipment is strategically placed within the controlled environment to minimize the chance for the equipment to come in proximity to or is inadvertently contaminated by the tissue specimen.

Next, the placenta is removed from the sterile shipment bag and transferred aseptically to a sterile processing basin within the controlled environment. The sterile basin contains hyperisotonic saline solution (e.g., 18% NaCl) that is at room or near room temperature. The placenta is gently massaged to help separate blood clots and to allow the placental tissue to reach room temperature, which will make the separation of the placental components from each other (e.g., amnion membrane and chorion). After having warmed up to the ambient temperature (after about 10-30 minutes), the placenta is then removed from the sterile processing basin and laid flat on a processing tray with the amnion membrane layer facing down for inspection.

The placenta tissue is examined for discoloration, debris or other contamination, odor, and signs of damage. The size of the tissue is also noted. A determination is made, at this point, as to whether the tissue is acceptable for further processing.

Next, if the placenta tissue is deemed acceptable for further processing, in one aspect, the amnion membrane and chorion of the placenta tissue are then carefully separated. In one aspect, the materials and equipment used in this procedure include a processing tray, 18% saline solution, sterile 4×4 sponges, and two sterile Nalgene jars. The placenta tissue is then closely examined to find an area (typically a corner) in which the amnion membrane can be separated from the chorion. The amnion membrane appears as a thin, opaque layer on the chorion.

The fibroblast layer is identified by gently contacting each side of the amnion membrane with a piece of sterile gauze or a cotton tipped applicator. The fibroblast layer will stick to the test material. The amnion membrane is placed into processing tray basement membrane layer down. Using a blunt instrument, a cell scraper or sterile gauze, any residual blood is also removed. This step must be done with adequate care, again, so as not to tear the amnion membrane. The cleaning of the amnion membrane is complete once the amnion membrane is smooth and opaque-white in appearance.

In certain aspects, the intermediate tissue layer is removed from the amnion membrane. This can be performed by peeling the intermediate tissue layer from the amnion membrane. Alternatively, the intermediate tissue layer can be removed from the amnion membrane by wiping the intermediate tissue layer with a gauze or other suitable wipe. The resulting amnion can be subsequently decontaminated using the process described below. The intermediate tissue layer does not require any additional processing and can be used as-is.

In certain aspects, the Wharton's jelly can optionally be isolated using the following procedure. Using a scalpel or scissors, the umbilical cord is dissected away from the chorionic disk. Once the veins and the artery have been identified, the cord is dissected lengthwise down one of the veins or the artery. Once the umbilical cord has been dissected, surgical scissors and forceps can be used to dissect the vein and artery walls from the Wharton's jelly. Next, the outer layer of amnion membrane is removed from the Wharton's jelly by cutting the amnion membrane. After removing the amnion membrane from the Wharton's jelly, the Wharton's jelly can be cut into strips. In one aspect, the strips are approximately 1-4 cm by 10-30 cm with an approximate thickness of 1.25 cm; however, other thicknesses are possible depending on application.

### Chemical Decontamination (Step 140)

Any of the placental components discussed herein can be chemically decontaminated using the techniques described below. In one aspect, the amnion membrane produced in step 130 is placed into a sterile Nalgene jar for the next step for additional cleaning In one aspect, the following procedure can be used to clean the amnion membrane. Each Nalgene jar is aseptically filled with 18% saline hypertonic solution and sealed (or sealed with a top). The jar is then placed on a rocker platform and agitated for between 30 and 90 minutes, which further cleans the tissue of contaminants. If the rocker platform was not in the critical environment (e.g., the manufacturing hood), the Nalgene jar is returned to the controlled/sterile environment and opened. Using sterile forceps or by aseptically decanting the contents, the tissue is gently removed from the Nalgene jar containing the 18% hyperisotonic saline solution and placed into an empty Nalgene jar. This empty Nalgene jar with the tissue is then aseptically filled with a pre-mixed antibiotic solution. Preferably, the premixed antibiotic solution is comprised of a cocktail of antibiotics, such as Streptomycin Sulfate and Gentamicin Sulfate. Other antibiotics, such as Polymixin B Sulfate and Bacitracin, or similar antibiotics now available or available in the future, are also suitable. Additionally, it is preferred that the antibiotic solution be at room temperature when added so that it does not change the temperature of or otherwise damage the tissue. This jar or container containing the tissue and antibiotics is then sealed or closed and placed on a rocker platform and agitated for, preferably, between 60 and 90 minutes. Such rocking or agitation of the tissue within the antibiotic solution further cleans the tissue of contaminants and bacteria. Optionally, the amnion membrane can be washed with a detergent. In one aspect, the amnion membrane can be washed with 0.1 to 10%, 0.1 to 5%, 0.1 to 1%, or 0.5% Triton-X wash solution.

Again, if the rocker platform was not in the critical environment (e.g., the manufacturing hood), the jar or container containing the tissue and antibiotics is then returned to the critical/sterile environment and opened. Using sterile forceps, the tissue is gently removed from the jar or container and placed in a sterile basin containing sterile water or normal saline (0.9% saline solution). The tissue is allowed to soak in place in the sterile water/normal saline solution for at least 10 to 15 minutes. The tissue may be slightly agitated to facilitate removal of the antibiotic solution and any other contaminants from the tissue. After at least 10 to 15 minutes, the tissue is ready to be dehydrated and processed further.

In the case when the chorion is to be used, the following exemplary procedure can be used. After separation of the chorion from the amnion membrane and removal of clotted blood from the fibrous layer, the chorion is rinsed in 18% saline solution for 15 minutes to 60 minutes. During the first rinse cycle, 18% saline is heated in a sterile container using a laboratory heating plate such that the solution temperature is approximately 48° C. The solution is decanted, the chorion tissue is placed into the sterile container, and decanted saline solution is poured into the container. The container is sealed and placed on a rocker plate and agitated for 15 minutes to 60 minutes. After 1 hour agitation bath, the chorion tissue was removed and placed into second heated agitation bath for an additional 15 minutes to 60 minutes rinse cycle. Optionally, the chorion tissue can be washed with a detergent (e.g., Triton-X wash solution) as discussed above for the amnion membrane. The container is sealed and agitated without heat 15 minutes to 120 minutes hours. The chorion tissue is next washed with deionized water (250 ml of DI water4) with vigorous motion for each rinse. The tissue is removed and placed into a container of 1× PBS w/EDTA solution. The container is sealed and agitated for 1 hour at controlled temperature for 8 hours. The chorion tissue is removed and rinsed using sterile water. A visual inspection was performed to remove any remaining discolored fibrous blood material from the chorion tissue. The chorion tissue should have a cream white visual appearance with no evidence of brownish discoloration.

In the case of Wharton's jelly, it can be transferred to a sterile Nalgene jar. Next, room temperature 18% hypertonic saline solution is added to rinse the tissue and the jar is sealed. The jar is agitated for 30 to 60 minutes. After incubation, the jar is decontaminated and returned to the sterile field. The tissue is transferred to a clean sterile Nalgene jar and prewarmed (about 48° C.) 18% NaCl is added. The container is sealed and placed on a rocker plate and agitated for 60 to 90 minutes.

In one aspect, after the rinse, the jar is decontaminated and returned to the sterile field. The tissue is removed and placed into an antibiotic solution. The container is sealed and agitated for 60 to 90 minutes on a rocker platform. Following incubation, the jar may be refrigerated at 1 to 10° C. for up to 24 hours. The Wharton's jelly is next transferred to a sterile basin containing approximately 200 mL of sterile water. The tissue is rinsed for 1-2 minutes and transferred to a sterile Nalgene jar containing approximately 300 ml of sterile water. The jar is sealed and placed on the rocker for 30 to 60 minutes. After the incubation, the jar is returned to the sterile field. The Wharton's jelly should have a cream white visual appearance with no evidence of brownish discoloration. The tissue is ready for further processing.

### Dehydration (Step 145)

One or more placental tissues prepared above can be dehydrated to produce dehydrated placental tissue grafts. In the case when two or more placental tissues are used, the tissue graft is a laminate. For example, a laminate composed of amnion, amnion membrane, chorion, Wharton's jelly, or any combination thereof can be produced. In one aspect, the tissue graft is an amnion/chorion laminate. In another aspect, the tissue has at least two layers of chorion, at least two layers of amnion, or at least one layer of chorion and amnion. In a further aspect, the placental tissue graft has a plurality chorion and/or amnion membranes laminated to one another. Techniques for producing laminated tissue grafts are know in the art.

In one aspect, the tissue graft is dehydrated by chemical dehydration followed by freeze-drying. In one aspect, the chemical dehydration step is performed by contacting the placental tissue with a polar organic solvent for a sufficient time and amount in order to substantially (i.e., greater than 90%, greater than 95%, or greater than 99%) or completely remove residual water present in the placental tissue (i.e., dehydrate the tissue). The solvent can be protic or aprotic. Examples of polar organic solvents useful herein include, but are not limited to, alcohols, ketones, ethers, aldehydes, or any combination thereof. Specific, non-limiting examples include DMSO, acetone, tetrahydrofuran, ethanol, isopropanol, or any combination thereof. In one aspect, the placental tissue is contacted with a polar organic solvent at room temperature. No additional steps are required, and the placental tissue can be freeze-dried directly as discussed below.

After chemical dehydration, the placental tissue graft is freeze-dried in order to remove any residual water and polar organic solvent. In one aspect, the placental tissue is placed in a freeze-dryer, and the placental tissue is lyophilized between −50° C. to 80° C. In another aspect, the placental tissue is placed in a freeze-dryer such that it is hanging in the freeze-dryer. In other aspects, the placental tissue is placed on a substrate that can facilitate free-drying. Examples of such substrates include, but are not limited to, a pan, bowl, screen, or a frame. In one aspect, one or more placental tissues can optionally be laid on a suitable drying fixture prior to freeze-drying. For example, at least two layers of hydrated chorion, at least two layers of hydrated amnion, or at least one layer of hydrated chorion and hydrated amnion can be applied to the drying fixture. In other aspects, the placental tissue composed of amnion membrane, Wharton's jelly, and chorion that has not been separated can be laid on top of the drying fixture, where one or more additional placental tissues such as amnion membrane and/or chorion can optionally be applied on top of the tissue.

The drying fixture is preferably sized to be large enough to receive the placental tissue, fully, in laid out, flat fashion. In one aspect, the drying fixture is made of Teflon or of Delrin, which is the brand name for an acetal resin engineering plastic invented and sold by DuPont and which is also available commercially from Werner Machine, Inc. in Marietta, Ga. Any other suitable material that is heat and cut resistant, capable of being formed into an appropriate shape to receive wet tissue can also be used for the drying fixture.

In one aspect, the receiving surface of the drying fixture can have grooves that define the product spaces, which are the desired outer contours of the tissue after it is cut and of a size and shape that is desired for the applicable surgical procedure in which the tissue will be used. For example, the drying fixture can be laid out so that the grooves are in a grid arrangement. The grids on a single drying fixture may be the same uniform size or may include multiple sizes that are designed for different surgical applications. Nevertheless, any size and shape arrangement can be used for the drying fixture, as will be appreciated by those skilled in the art. In another aspect, instead of having grooves to define the product spaces, the drying fixture has raised ridges or blades.

In certain aspects, the drying fixture can include a slightly raised or indented texture in the form of text, logo, name, or similar design. This textured text, logo, name, or design can be customized or private labeled depending upon the company that will be selling the graft or depending upon the desired attributes requested by the end user (e.g., surgeon). When dried, the tissue will mold itself around the raised texture or into the indented texture, essentially providing a label within the tissue itself. Preferably, the texture/label can be read or viewed on the placental tissue in only one orientation so that, after dehydration, an end user (e.g., a surgeon) of the dried tissue will be able to identify the top and bottom of the placental tissue. In other aspects, a stamp can be imprinted on the placental tissue graft after freeze-drying in order to differentiate the sides of the graft.

Once the placental tissue(s) is placed on the drying fixture, the drying fixture is placed in the freeze-dryer. The use of the freeze-dryer to dehydrate the placental tissue grafts can be more efficient and thorough compared to other techniques such as thermal dehydration. In general, it is desirable to avoid ice crystal formation in the placental tissue grafts as this may damage the extracellular matrix in the tissue graft. By chemically dehydrating the placental tissue prior to freeze-drying, this problem can be avoided.

In another aspect, the dehydration step involves applying heat to the placental tissue. In one aspect, the placental component is laid on a suitable drying fixture as discussed above, and the drying fixture is placed in a sterile Tyvex (or similar, breathable, heat-resistant, and sealable material) dehydration bag and sealed. The breathable dehydration bag prevents the tissue from drying too quickly. If multiple drying fixtures are being processed simultaneously, each drying fixture is either placed in its own Tyvex bag or, alternatively, placed into a suitable mounting frame that is designed to hold multiple drying frames thereon and the entire frame is then placed into a larger, single sterile Tyvex dehydration bag and sealed.

The Tyvex dehydration bag containing the one or more drying fixtures is then placed into a non-vacuum oven or incubator that has been preheated to approximately 35 to 50 degrees Celcius. The Tyvex bag remains in the oven for between 30 and 120 minutes, although approximately 45 minutes at a temperature of approximately 45 degrees Celcius appears to be ideal to dry the tissue sufficiently but without over-drying or burning the tissue. The specific temperature and time for any specific oven will need to be calibrated and adjusted based on other factors including altitude, size of the oven, accuracy of the oven temperature, material used for the drying fixture, number of drying fixtures being dried simultaneously, whether a single or multiple frames of drying fixtures are dried simultaneously, and the like.

### Preparation of Shredded Tissue Grafts (Step 150)

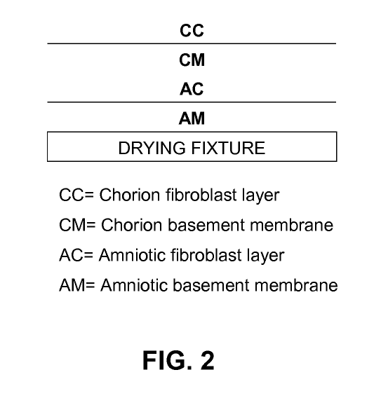

Once the placental components (e.g., amnion membrane or amnion, intermediate tissue layer, chorion, Wharton's jelly, placental disk) have been isolated, decontaminated and dehydrated, the placental components are shredded to produce shredded tissue grafts. The selection of placental components used to make the shredded tissue grafts can vary depending upon the application of the shredded tissue graft. In one aspect, the shredded tissue grafts are composed of 100% amnion membrane, chorion, or Wharton's jelly. In one aspect, the shredded tissue graft can be composed of chorion laminated to amnion membrane. In another aspect, the shredded tissue graft comprises (1) an amnion membrane comprising an amniotic basement membrane and an amniotic fibroblast layer, and (2) a chorion membrane comprising a chorion basement membrane and a chorion fibroblast layer, wherein the chorion basement membrane is adjacent to the amniotic fibroblast layer. This aspect is depicted in FIG. 2.

The following exemplary procedure can be used to produce the shredded tissue grafts. The tissue grafts prepared above are introduced into a tissue processor, where the processor has two blades constructed out of two pins that are in close proximity to one another. The blades are notched with alternating notches such that the notches on one blade align with the grooves on the other blade. Each notch is spaced approximately 2 mm apart. The tissue graft is fed perpendicular to the blades and comes out on the opposite side to produce the shredded tissue graft. The dimensions of the shredded tissue graft can vary depending upon the end-use of the graft. In one aspect, the shredded tissue graft has an average length of 1 cm to 10 cm and an average width of 1 cm to 5 cm. In another aspect the shredded tissue graft has an average length of 2 cm, 6 cm, or 8 cm and average width of 1.5 cm, 2 cm, or 3 cm. In a further aspect, the shredded tissue graft has the following length and width dimensions: 8 cm×1 cm, 8 cm×2 cm, 8 cm×3 cm, 8 cm×4 cm, 8 cm×5 cm, 8 cm×6 cm, 8 cm×7 cm, 8 cm×8 cm, 6 cm×1 cm, 6 cm×2 cm, 6 cm×3 cm, 6 cm×4 cm, 6 cm×5 cm, 6 cm×6 cm, 4 cm×1 cm, 4 cm×2 cm, 4 cm×3 cm, or 4 cm×4 cm. The thickness of the shredded tissue grafts can vary as well. In one aspect, the hydrated shredded tissue grafts have a thickness from 0.025 mm to 0.500 mm, 0.050 mm to 0.300 mm, 0.100 mm to 0.200 mm, 0.125 mm to 0.175 mm, or about 0.150 mm. In another aspect, the dehydrated shredded tissue grafts have a thickness from 0.010 mm to 0.300 mm, 0.025 mm to 0.200 mm, 0.050 mm to 0.100 mm, or 0.075 mm to 0.090 mm.

In another aspect, one or more bioactive agents can be added to the tissue grafts prior to and/or after shredding. In one aspect, the tissue graft before and/or after shredding can be soaked in a solution containing one or more bioactive agents. Examples of bioactive agents include, but are not limited to, naturally occurring growth factors sourced from platelet concentrates, either using autologous blood collection and separation products, or platelet concentrates sourced from expired banked blood; bone marrow aspirate; stem cells derived from concentrated human placental cord blood stem cells, concentrated amniotic fluid stem cells or stem cells grown in a bioreactor; or antibiotics. Upon application of the shredded tissue graft with bioactive agent to the region of interest, the bioactive agent can be delivered to the region over time.

In certain aspects, the placental component(s) can be cross-linked with one another. For example, a cross-linking agent can be applied to the tissue graft prior to and/or after shredding. In general, the cross-linking agent is nontoxic and non-immunogenic. In one aspect, when the amnion membrane or amnion, intermediate tissue layer, and chorion are treated with the cross-linking agent, the cross-linking agent can be the same or different. In another aspect, the amnion membrane or amnion, intermediate tissue layer, and chorion can be treated separately with a cross-linking agent or, in the alternative, the amnion membrane or amnion, intermediate tissue layer, and chorion can be treated together with the same cross-linking agent. In certain aspects, the amnion membrane or amnion, intermediate tissue layer, and chorion can be treated with two or more different cross-linking agents. The conditions for treating the amnion membrane or amnion, intermediate tissue layer, and chorion can vary. In one aspect, the concentration of the cross-linking agent is from 0.1 M to 5 M, 0.1 M to 4 M, 0.1 M to 3 M, 0.1 M to 2 M, or 0.1 M to 1 M.

The cross-linking agent generally possesses two or more functional groups capable of reacting with proteins to produce covalent bonds. In one aspect, the cross-linking agent possesses groups that can react with amino groups on proteins present in the amnion, intermediate tissue layer, and chorion. Examples of such functional groups include, but are not limited to, hydroxyl groups, substituted or unsubstituted amino groups, carboxyl groups, and aldehyde groups. In one aspect, the cross-linker can be a dialdehydes such as, for example, glutaraldehyde. In another aspect, the cross-linker can be a carbodiimide.

In one aspect, sugar is the cross-linking agent, where the sugar can react with proteins present in the amnion, intermediate tissue layer, and chorion to form a covalent bond. For example, the sugar can react with proteins by the Maillard reaction, which is initiated by the nonenzymatic glycosylation of amino groups on proteins by reducing sugars and leads to the subsequent formation of covalent bonds. Examples of sugars useful as cross-linking agent include, but are not limited to, D-ribose, glycerone, altrose, talose, ertheose, glucose, lyxose, mannose, xylose, gulose, arabinose, idose, allose, galactose, maltose, lactose, sucrose, cellibiose, gentibiose, melibiose, turanose, trehalose, isomaltose, or any combination thereof.

### II. Applications of Shredded Tissue Grafts

The shredded tissue grafts described herein have numerous applications and can be used in a variety of procedures. In one aspect, the shredded tissue grafts described herein are useful in enhancing or improving wound healing. The types of wounds that present themselves to physicians on a daily bases are diverse. Acute wounds are caused by surgical intervention, trauma and burns. Chronic wounds are wounds that are delayed in closing compared to healing in an otherwise healthy individual. Examples of chronic wound types plaguing patients include diabetic foot ulcers, venous leg ulcers, pressure ulcers, arterial ulcers, and surgical wounds that become infected as well as non-diabetic wounds such as, for example, dermal wounds caused by cancer therapy (radiation, chemotherapy). Here, the shredded tissue grafts described herein are useful in wounds where there is deep tissue damage and exposure of the musculature, tendons, ligaments, or bone.

The physician's goal when treating traumatic wounds is to heal the wound while allowing the patient to retain natural function in the area of the wound with minimal scaring and infection. If a wound becomes infected, it can lead to a loss of limb or life. For the most part, physicians heal these patients without incident. However, physicians dealing with chronic wounds are mainly concerned with closing the wound as quickly as possible to minimize the risk of an infection that could lead to loss of limb or life. Chronic wounds are wounds on patients that have comorbidities that complicate or delay the healing cascade. In one aspect, the grafts described herein can function as a tissue regeneration template that delivers essential wound healing factors, extracellular matrix proteins and inflammatory mediators to help reduce inflammation, enhance healing, and reduces scar tissue formation.

In one aspect, the shredded tissue grafts can be used as a bulk dressing or filler. In this aspect, the shredded tissue graft is useful in filling a void or hole in a subject in order to enhance wound healing. In other aspects, the shredded tissue grafts are effective in healing dermal wounds, where the wound is present in the dermis and/or subdermis. Thus, the shredded tissue grafts are effective in treatment of deep skin wounds. The source of the dermal wound can vary. In one aspect, the shredded tissue grafts can be used to heal an acute, open wound including, but not limited to, an incision, a laceration, an abrasion, a puncture, or a burn. In other aspects, the shredded tissue grafts can be used to heal a chronic wound such as a diabetic ulcer, a venous ulcer, or a pressure ulcer.

The shredded tissue grafts are more effective than other dressings with respect to wound healing. The shredded tissue grafts are capable of absorbing physiological fluids once placed in the open wound. The ability of the grafts to absorb fluids makes them hemostatic agents, where the grafts can facilitate clotting with blood and other physiological fluids in the wound. This feature can further enhance wound healing. In addition to the advantages discussed above, the ability of the shredded tissue grafts to absorb fluids permits them to be admixed with a variety of substances (e.g., any of the bioactive agents described herein) to produce pharmaceutical compositions with enhanced activity. For example, the shredded tissue grafts can be mixed with additional hemostatic agents to enhance blood clotting at a wound.

The shredded tissue grafts also minimize or prevent the chance of infection in the wound and subsequent inflammation. Symptoms of wound infection include redness, pain, heat, and swelling of the wound and periwound area. In one aspect, the shredded tissue grafts can reduce the risk of post-operative wound infection by maintaining a biological protective barrier in the wound for greater than 48 hours. Additionally, the shredded tissue grafts do not illicit and immune response or allergic reaction. Finally, the shredded tissue grafts contain growth factors that promote healing and reduce the chance of inflammation.

Not wishing to be bound by theory, the shredded tissue graft can enhance healing in both primary and secondary wound intention. In this aspect, wound healing happens from the inside out. The shredded tissue grafts improve tensile strength at the site of the wound and assist in collagen fiber reorganization. Moreover, the shredded tissue grafts provide a natural non-adherent lattice for reducing excisional surgical pain. Here, the graft provides a three-dimensional lattice or scaffold for tissue growth. The shredded tissue graft when placed into the wound reduces the incidence of wound dehiscence by influencing soft tissue healing by the introduction of growth factors in the wound. Thus, the shredded tissue graft prevents the wound from re-opening.

The shredded tissue grafts also provide increased skin re-apposition during healing by primary intention. Thus, the shredded tissue grafts also prevent or reduce scar formation because the grafts serve as a reduced friction anatomical barrier. This is particularly relevant in wounds caused by repeated surgical procedures at the same location.

In other aspects, the shredded tissue grafts can be admixed with fibrin glues to enhance wound healing. The shredded tissue grafts can enhance the ability of the fibrin glue to form fibrin clots and enhance tissue repair. Thus, the shredded tissue grafts in combination with the fibrin glue can further reduce the need of sutures typically used to close the wound, which ultimately reduces the risk of infection, inflammation, and scar formation at the wound.

Various modifications and variations can be made to the shredded tissue grafts and methods described herein. Other aspects of the compounds, compositions and methods described herein will be apparent from consideration of the specification and practice of the compounds, compositions and methods disclosed herein. It is intended that the specification and examples be considered as exemplary.

1. A shredded tissue graft comprising one or more placental components.

2. The graft of claim 1, wherein the placental components comprise amnion membrane, amnion, chorion, Wharton's jelly, placental disk, or any combination thereof.

3. The graft of claim 1, wherein the graft comprises chorion laminated to an amnion membrane.

4. The graft of claim 1, wherein graft comprises
(1) an amnion membrane comprising an amniotic basement membrane and an amniotic fibroblast layer, and
(2) a chorion membrane comprising a chorion basement membrane and a chorion fibroblast layer, wherein the chorion basement membrane is adjacent to the amniotic fibroblast layer.

5. The graft in claim 1, wherein the graft has an average length of 1 cm to 10 cm and an average width of 1 cm to 5 cm.

6. The graft in claim 1, wherein graft further comprises one or more bioactive agents.

7. A method for enhancing the healing of a dermal wound in a subject, the method comprising applying the graft in claim 1 in the wound.

8. The method of claim 7, wherein the wound is an acute, open wound.

9. The method of claim 8, wherein the acute, open wound is an incision, a laceration, an abrasion, a puncture, or a burn.

10. The method of claim 7, wherein the wound is a chronic wound.

11. The method of claim 10, wherein the chronic wound is a diabetic ulcer, a venous ulcer, or a pressure ulcer.

12. The method in claim 7, wherein the graft enhances wound hemostasis in the wound.

13. The method in claim 7, wherein the graft reduces or prevents inflammation in and/or near the wound.

14. The method in claim 7, wherein the graft reduces or prevents infection in and/or near the wound.

15. The method in claim 7, wherein the graft reduces or prevents scar formation at the wound.

16. The use of the graft in claim 7 for filling a void in a dermal wound.
